(19) United States
(12) Patent Application Publication
(10) Pub. No.: US 2011/0196158 A1
(43) Pub. Date: Aug. 11, 2011

(54) TRIS(ARYLBENZOXAZOLE)BENZENE AND TRIS(ARYLBENZTHIAZOLE)BENZENE AND DERIVATIVES THEREOF AS ORGANIC ELECTRON-TRANSPORT MATERIALS

(76) Inventor: Shijun Zheng, San Diego, CA (US)

(21) Appl. No.: 13/020,782

(22) Filed: Feb. 3, 2011

Related U.S. Application Data
(60) Provisional application No. 61/301,711, filed on Feb. 5, 2010.

(51) Int. Cl. C07D 417/14; C07D 413/14

(52) U.S. Cl. 546/256; 548/156; 548/219

(57) ABSTRACT

A compound represented by a Formula 1:

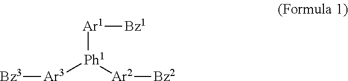

wherein Ph¹, Ar¹, Ar², Ar³ Bz¹, Bz², and Bz³ are described herein. Light-emitting devices containing the compound of Formula are also described herein.

### CROSS-REFERENCE TO RELATED APPLICATIONS

This application claims the benefit of U.S. Provisional Application No. 61/301,711, filed Feb. 5, 2010, which is incorporated by reference herein in its entirety.

### BACKGROUND

1. Field

The embodiments relate to compounds for light-emitting devices, such as compounds useful as electron-transport materials.

2. Description of the Related Art

Organic light-emitting devices (OLEDs) have been widely developed for flat panel displays, and are moving fast towards solid state lighting (SSL) applications. Some believe that a white OLED device with greater than 1,500 lm, a color rendering index (CRI) greater than 70, and an operating time greater than 10,000 hours at 1,000 lm/w may be useful in SSL applications. In order to reduce the driving voltage of an OLED device and extend the operational lifetime, it may be helpful to develop new high performance electron transport materials.

In organic electroluminescence (EL) devices, charges injected from both electrodes recombine in the emissive layer to thereby provide light emission. However, in many cases, the mobility of holes is higher than the electron mobility, which may cause some holes to partially pass beyond the emissive layer before recombining with electrons. For example, Tris(8-hydroxyquinoline) aluminum (Alq₃) is a common electron transport material, but its electron mobility is lower than the hole mobility of some hole-transport materials. Since recombination of a hole and an electron outside of the emissive layer does not result in light emission, use of Alq₃ with these higher hole mobility hole-transport materials may cause a reduction of efficiency for the device. For this reason, there is a demand for electron transport materials having higher electron mobility.

Responsive to this demand, several compounds having been developed which have increased electron mobility such as 2-(4-biphenylyl)-5-(4-t-butylphenyl)-1,3,4-oxadiazole (PBD) and 2 2′,2″-(1,3,5-phenylene)tris[1-phenyl-1H-benzimidazole (TPBI). However, these compounds may not be as stable as desired in a thin film state, perhaps because of undesired crystallization. To overcome this stability problem, additional electron transporting materials such as several benzimidazole substituted analogues have been described (U.S. Pat. No. 6,171,715; JP2002212181A; “Pyridine-Containing Triphenylbenzene Derivatives with High Electron Mobility for Highly Efficient Phosphorescent OLEDs”, Su, Shi-Jian, et al, Adv. Mater. (2008) 20(11):2125-2130) and various oxadiazole derivatives have been proposed. While these electron transport materials have been improved in stability compared with PBD, the improvement is often not considered to be sufficient. For these reasons, Alq3 is still used, even with hole-transport materials having a hole mobility which is higher than the electron mobility of Alq₃. Thus, there is room to improve OLEDs by improving the electron mobility of electron transport materials.

Another approach to improving device efficiency is to insert a hole blocking layer next to the emissive layer. This approach seeks to reduce the relative number of holes which can pass through the emissive layer, and thus improve the probability of charge recombination in the emissive layer. Some examples of hole blocking materials known in the art include triazole derivatives, bathocuproine (BCP), and mixed-ligand complexes of aluminum (e.g. BAlq). However, these materials may still have less than desirable film stability or hole blocking. Thus, satisfactory device characteristics have not been obtained.

Thus there is a need for additional organic compounds which provide improved electron injection/transport performance and/or hole blocking abilities while maintaining high stability in a thin film state in order to improve the device characteristics of organic EL devices.

### SUMMARY

Some embodiments are related to a compound represented by a formula:

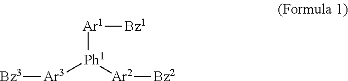

wherein Ph¹ is optionally substituted phenyl; Ar¹, Ar², and Ar³ are independently optionally substituted m-phenylene or m-pyridinylene; and Bz¹, Bz², and Bz³ are independently optionally substituted benzooxazol-2-yl or benzothiazol-2-yl.

Some embodiments provide an organic light-emitting device comprising an organic component disposed between an anode and a cathode, wherein the organic component comprises a compound described herein. In some embodiments, the organic component further comprises at least one layer comprising the compound, wherein the layer is selected from: an electron-transport layer, an electron-injecting layer, and an electron-injecting and electron-transport layer.

These and other embodiments are described in greater detail below.

### BRIEF DESCRIPTION OF THE DRAWINGS

FIG. 1 depicts an exemplary configuration of an embodiment of a device described herein.

FIG. 2 shows the electroluminescence spectrum of an embodiment of a device of FIG. 1.

FIG. 3 shows current density and luminance as a function of the driving voltage of an embodiment of a device of FIG. 1.

FIG. 4 shows the device current and power efficiency as a function of device brightness of an embodiment of a device of FIG. 1.

### DETAILED DESCRIPTION OF SOME EMBODIMENTS

Unless otherwise indicated, when a chemical structural feature such as phenyl is referred to as being “optionally substituted,” it is meant that the feature may have no substituents (i.e. be unsubstituted) or may have one or more substituents. The term “optionally substituted” may apply to any position on a structural feature where there is no specific linkage to another moiety. A feature that is “substituted” has one or more substituents. The term “substituent” has the ordinary meaning known to one of ordinary skill in the art. In some embodiments, the substituent is an ordinary organic moiety known in the art, which may have a molecular weight (e.g. the sum of the atomic masses of the atoms of the substituent) of less than: about 500 g/mol, about 300 g/mol, about 200 g/mol, about 100 g/mol, or about 50 g/mol. In some embodiments, the substituent comprises: about 0-30, about 0-20, about 0-10, or about 0-5 carbon atoms; and about 0-30, about 0-20, about 0-10, or about 0-5 heteroatoms independently selected from: N, O, S, P, Si, F, Cl, Br, I, and combinations thereof; provided that the substituent comprises at least one atom selected from: C, N, O, S, P, Si, F, Cl, Br, and I. Examples of substituents include, but are not limited to, alkyl, alkenyl, alkynyl, carbazolyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, and amino, including mono- and di-substituted amino groups, and the protected derivatives thereof.

As used herein the term “aryl” has the ordinary meaning understood by a person of ordinary skill in the art. In some embodiments, the term “phenyl” refers to any optionally substituted ring, including those which attach to the remaining portion of the molecule in 6 positions, 5 positions, 4 positions, 3 positions, 2 positions, or 1 position. In some embodiments, the phenyl is phenylene, such as m-phenylene. In some embodiments, the phenyl may have 0, 1, 2, 3, or 4 substituents independently selected from: C₁₋₆ alkyl, such as methyl, ethyl, propyl isomers, cyclopropyl, butyl isomers, cyclobutyl isomers (such as cyclobutyl, methylcyclopropyl, etc.), pentyl isomers, cyclopentyl isomers, hexyl isomers, cyclohexyl isomers, etc.; optionally substituted phenyl; —OR′; —COR′; —CO₂R′; —OCOR′; —NR′COR″; CONR′R″; —NR′R″; F; Cl; Br; I; nitro; CN, etc.; wherein R′ and R″ are independently H, optionally substituted phenyl, or C₁₋₆ alkyl.

As used herein, the term “alkyl” refers to a moiety composed of carbon and hydrogen containing no double or triple bonds. Alkyl may be linear, branched, cyclic, or a combination thereof, may be bonded to any other number of moieties (e.g. be bonded to 1 other group, such as —CH₃, 2 other groups, such as —CH₂—, or any number of other groups) that the structure may bear, and in some embodiments, may contain from one to thirty-five carbon atoms. Examples of alkyl groups include but are not limited to CH₃ (e.g. methyl), C₂H₅ (e.g. ethyl), C₃H₇ (e.g. propyl isomers such as propyl, isopropyl, etc.), C₃H₆ (e.g. cyclopropyl), C₄H₉ (e.g. butyl isomers) C₄H₈ (e.g. cyclobutyl isomers such as cyclobutyl, methylcyclopropyl, etc.), C₅H₁₁ (e.g. pentyl isomers), C₅H₁₀ (e.g. cyclopentyl isomers such as cyclopentyl, methylcyclobutyl, dimethylcyclopropyl, etc.) C₆H₁₃ (e.g. hexyl isomers), C₆H₁₂ (e.g. cyclohexyl isomers), C₇H₁₅ (e.g. heptyl isomers), C₇H₁₄ (e.g. cycloheptyl isomers), C₈H₁₇ (e.g. octyl isomers), C₈H₁₆ (e.g. cyclooctyl isomers), C₉H₁₉ (e.g. nonyl isomers), C₉H₁₈ (e.g. cyclononyl isomers), C₁₀H₂₁ (e.g. decyl isomers), C₁₀H₂₀ (e.g. cyclodecyl isomers), C₁₁H₂₃ (e.g. undecyl isomers), C₁₁H₂₂ (e.g. cycloundecyl isomers), C₁₂H₂₅ (e.g. dodecyl isomers), C₁₂H₂₄ (e.g. cyclododecyl isomers), C₁₃H₂₇ (e.g. tridecyl isomers), C₁₃H₂₆ (e.g. cyclotridecyl isomers), and the like.

As used herein, the term “alkoxy” refers to —O-alkyl, such as —OCH₃, —OC₂H₅, —OC₃H₇ (e.g. propoxy isomers such as isopropoxy, n-propoxy, etc.), —OC₄H₉ (e.g. butyoxy isomers), —OC₅H₁₁ (e.g. pentoxy isomers), —OC₆H₁₃ (e.g. hexoxy isomers), —OC₇H₁₅ (e.g. heptoxy isomers), etc.

As used herein, the term “haloalkyl” refers to alkyl having one or more halo substituents. For example, the term “fluoroalkyl” refers to alkyl having one or more fluoro substituents. The term “perfluoroalkyl” refers to fluoroalkyl wherein all hydrogen atom are replaced by fluoro such as —CF₃, —C₂F₅, —C₃F₇, —C₄F₉, etc.

Generally, an expression such as “C₆₋₁₀” (e.g. “C₆₋₁₀ aryl”) refers only to the number of carbon atoms in a parent group, and does not characterize or limit the substituents in any way. If any doubt arises as to whether a structural feature is a substituent or a parent group, the carbon atoms should be counted as if the structural feature is part of the parent group. For example, the carbon atoms of an alkyl “substituent” on an alkyl parent should be counted as part of the parent group.

The structures and names of some of the ring systems referred to herein are depicted below. If optionally substituted, these ring systems may be unsubstituted, as shown below, or a substituent may independently be in any position normally occupied by a hydrogen atom.

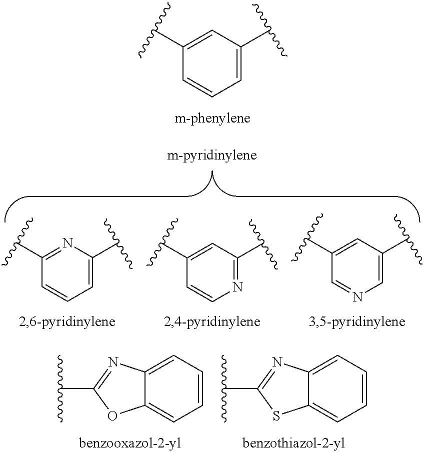

With respect to m-pyridinylene, the term encompasses several isomers, three of which are depicted above.

The term “low work function” has the ordinary meaning known to one of ordinary skill in the art. In some embodiments, a “work function” of a metal is a measure of the minimum energy required to extract an electron from the surface of the metal.

The term “high work function” has the ordinary meaning known to one of ordinary skill in the art. In some embodiments, a “high work function metal” is a metal or alloy that easily injects holes and typically has a work function greater than or equal to 4.5.

The term “low work function metal” has the ordinary meaning known to one of ordinary skill in the art. In some embodiments, a “low work function metal” is a metal or alloy that easily loses electrons and typically has a work function less than 4.3.

The expression “white light-emitting” has the ordinary meaning known to one of ordinary skill in the art. In some embodiments, a material is white light-emitting if it emits white light. In some embodiments, white light is light having the approximate CIE color coordinates (X=⅓, Y=⅓). The CIE color coordinates (X=⅓, Y=⅓) may be defined as the achromatic point. The X and Y color coordinates may be weights applied to the CIE primaries to match a color. A more detailed description of these terms may be found in CIE 1971, International Commission on Illumination, Colorimetry: Official Recommendations of the International Commission on Illumination, Publication CIE No. 15 (E-1.3.1) 1971, Bureau Central de la CIE, Paris, 1971 and in F. W. Billmeyer, Jr., M. Saltzman, Principles of Color Technology, 2nd edition, John Wiley & Sons, Inc., New York, 1981, both of which are hereby incorporated by reference in their entireties. The color rendering index (CRI) refers to the ability to render various colors and has values ranging from 0 to 100, with 100 being the best.

With respect to Formula 1, Ph¹ is optionally substituted phenyl. In some embodiments, Ph¹ may be unsubstituted, or may have 1, 2, or 3 substituents independently selected from: R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Ph¹ has 1 or 2 substituents independently selected from C₁₋₆ alkyl, OH, and C₁₋₆ alkoxy. In some embodiments, Ph¹ is unsubstituted. For any of these embodiments, Ph¹ may have a 1, 3, 5-substitution pattern, meaning that if the carbons on the ring are consecutively numbered from 1 to 6, Ar¹, Ar², and Ar³ would be attached to the carbons numbered 1, 3, and 5, such as in the structure shown in Formula 2.

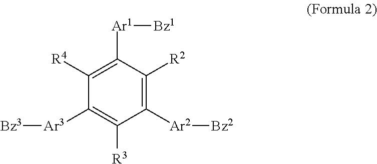

With respect to Formula 2, R², R³, and R⁴ may be any substituent. In some embodiments, R², R³, and R⁴ may independently be selected from the group consisting of: H, R^{o}, —OR¹, —OC(═O)R¹, —CO₂R¹, —(C═O)R¹, —NR²(C═O)R¹, —C(═O)NR¹R², —NR¹R², F, Cl, Br, and I. In some embodiments, R², R³, and R⁴ may independently be C₁₋₆ alkyl or C₁₋₆ alkoxy.

With respect to any embodiment, each R^{o} may independently be optionally substituted C₁₋₁₂ alkyl, such as unsubstituted alkyl or haloalkyl. In some embodiments, each R^{o} may be C₁₋₆ alkyl; such as methyl, ethyl, propyl isomers, cyclopropyl, butyl isomers, cyclobutyl isomers (such as cyclobutyl, methylcyclopropyl, etc.), pentyl isomers, cyclopentyl isomers, hexyl isomers, cyclohexyl isomers, etc.; or perfluoroalkyl such as CF₃, —C₂F₅, —C₃F₇, etc. Also with respect to any embodiment, each R¹ and each R² may independently be H or R^{o}.

With respect to Formula 1 or Formula 2, Ar¹ is optionally substituted m-phenylene or m-pyridinylene. In some embodiments, Ar¹ may have 0, 1, 2, 3, or 4 substituents. In some embodiments, any substituent of Ar¹ may independently be R^{o}, —OR¹, —OC(═O)R¹, —CO₂R¹, —(C═O)R¹, —NR²(C═O)R¹, —C(═)NR¹R², —NR¹R², F, Cl, Br, or I. In some embodiments, Ar¹ is 3,5-pyridinylene or m-phenylene, and has 1 or 2 substituents independently selected from C₁₋₆ alkyl and C₁₋₆ alkoxy. In some embodiments, Ar¹ is unsubstituted 3,5-pyridinylene or unsubstituted m-phenylene.

In embodiments related to Formula 1 or Formula 2, Ar² is optionally substituted m-phenylene or m-pyridinylene. In some embodiments, Ar² may have 0, 1, 2, 3, or 4 substituents. In some embodiments, any substituent of Ar² may independently be R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Ar² is 3,5-pyridinylene or m-phenylene, and has 1 or 2 substituents independently selected from C₁₋₆ alkyl and C₁₋₆ alkoxy. In some embodiments, Ar² is unsubstituted 3,5-pyridinylene or unsubstituted m-phenylene.

With respect to Formula 1 or Formula 2, Ar³ is optionally substituted m-phenylene or m-pyridinylene. In some embodiments, Ar³ may have 0, 1, 2, 3, or 4 substituents. In some embodiments, any substituent of Ar³ may independently be R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Ar³ is 3,5-pyridinylene or m-phenylene, and has 1 or 2 substituents independently selected from C₁₋₆ alkyl and C₁₋₆ alkoxy. In some embodiments, Ar³ is unsubstituted 3,5-pyridinylene or unsubstituted m-phenylene.

In embodiments related to Formula 1 or Formula 2, Bz¹ is optionally substituted benzooxazol-2-yl or benzothiazol-2-yl. In some embodiments, Bz¹ may have 0, 1, 2, 3, or 4 substituents. In some embodiments, any substituent of Bz¹ may independently be R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Bz¹ is benzooxazol-2-yl or benzothiazol-2-yl, and has 1 or 2 substituents independently selected from C₁₋₆ alkyl and C₁₋₆ alkoxy. In some embodiments, Bz¹ is unsubstituted benzooxazol-2-yl or unsubstituted benzothiazol-2-yl.

With respect to Formula 1 or Formula 2, Bz² is optionally substituted benzooxazol-2-yl or benzothiazol-2-yl. In some embodiments, Bz² may have 0, 1, 2, 3, or 4 substituents. In some embodiments, any substituent of Bz² may independently be R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Bz² is benzooxazol-2-yl or benzothiazol-2-yl, and has 1 or 2 substituents independently selected from C₁₋₆ alkyl and C₁₋₆ alkoxy. In some embodiments, Bz² is unsubstituted benzooxazol-2-yl or unsubstituted benzothiazol-2-yl.

In embodiments related to Formula 1 or Formula 2, Bz³ is optionally substituted benzooxazol-2-yl or benzothiazol-2-yl. In some embodiments, Bz³ may have 0, 1, 2, 3, or 4 substituents. In some embodiments, any substituent of Bz³ may independently be R^{o}, —OR¹, —OC(═O)R¹, —CO₂R¹, —(C═O)R¹, —NR²(C═O)R¹, —C(═O)NR¹R², —NR¹R², F, Cl, Br, or I. In some embodiments, Bz³ is benzooxazol-2-yl or benzothiazol-2-yl, and has 1 or 2 substituents independently selected from C₁₋₆ alkyl and C₁₋₆ alkoxy. In some embodiments, Bz³ is unsubstituted benzooxazol-2-yl or unsubstituted benzothiazol-2-yl.

In some embodiments related to Formula 1 or Formula 2, Ar¹, Ar², and Ar³ are optionally substituted m-phenylene; and Bz¹, Bz², and Bz³ are optionally substituted benzooxazol-2-yl. In some embodiments Ar¹, Ar², and Ar³ are optionally substituted m-phenylene; and Bz¹, Bz², and Bz³ are optionally substituted benzothiazol-2-yl. In other embodiments, Ar¹, Ar², and Ar³ are optionally substituted 3,5-pyridinylene; and Bz¹, Bz², and Bz³ are optionally substituted benzooxazol-2-yl. In other embodiments, Ar¹, Ar², and Ar³ are optionally substituted 3,5-pyridinylene; and Bz¹, Bz², and Bz³ are optionally substituted benzothiazol-2-yl. In embodiments where Ar¹, Ar², and Ar³ are optionally substituted m-phenylene, each m-phenylene may independently be unsubstituted, or have 1, 2, 3, or 4 substituents independently selected from R^{o}, —OR¹, —OC(═O)R¹, —CO₂R¹, —(C═O)R¹, —NR²(C═O)R¹, —C(═O)NR¹R², —NR¹R², F, Cl, Br, and I. In embodiments where Ar¹, Ar², and Ar³ are optionally substituted 3,5-pyridinylene, each 3,5-pyridinylene may independently be unsubstituted, or have 1, 2, or 3 substituents independently selected from R^{o}, —OR¹, —OC(═O)R¹, —CO₂R¹, —(C═O)R¹, —NR²(C═O)R¹, —C(═O)NR¹R², —NR¹R², F, Cl, Br, and I. In these embodiments, Bz¹, Bz², and Bz³ may be unsubstituted, or may have 1, 2, 3, or 4 substituents independently selected from R^{o}, —OR¹, —OC(═O)R¹, —CO₂R¹, —(C═O)R¹, —NR²(C═O)R¹, —C(═O)NR¹R², —NR¹R², F, Cl, Br, and I. In some of these embodiments, Ar¹; Ar¹ and Ar²; or Ar¹, Ar², and Ar³ are unsubstituted. In some of these embodiments, Bz¹; Bz¹ and Bz²; or Bz¹, Bz², and Bz³ are unsubstituted.

Some embodiments provide one of the compounds shown below:

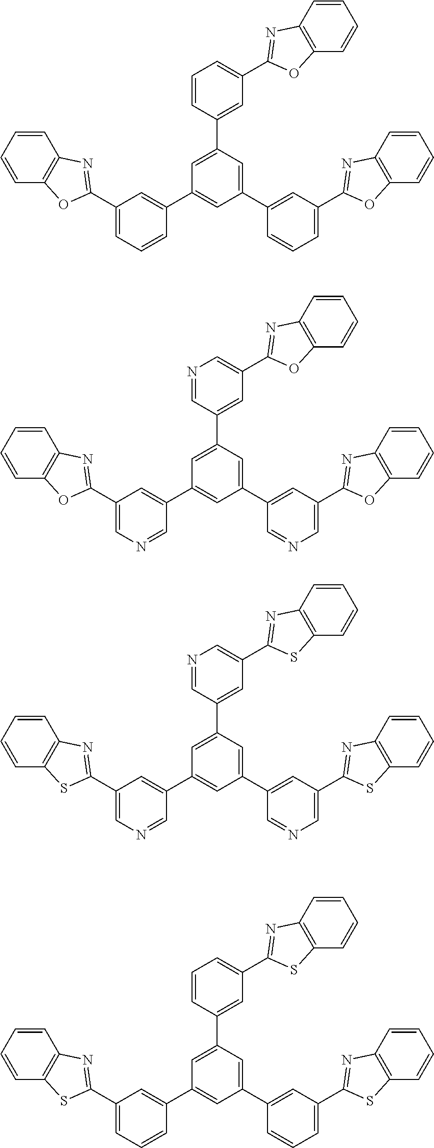

The compounds and compositions described herein can be incorporated into light-emitting devices in various ways. For example, an embodiment provides an organic component disposed between an anode and a cathode. In some embodiments, the device is configured so that holes can be transferred from the anode to the organic component. In some embodiments, the device is configured so that electrons can be transferred from the cathode to the organic component. The organic component comprises the compounds and/or compositions described herein.

The anode may be a layer comprising a conventional material such as a metal, mixed metal, alloy, metal oxide or mixed-metal oxide, conductive polymer, and/or an inorganic material such as carbon nanotube (CNT). Examples of suitable metals include the Group 1 metals, the metals in Groups 4, 5, 6, and the Group 8-10 transition metals. If the anode layer is to be light-transmitting, metals in Group 10 and 11, such as Au, Pt, and Ag, or alloys thereof; or mixed-metal oxides of Group 12, 13, and 14 metals, such as indium-tin-oxide (ITO), indium-zinc-oxide (IZO), and the like, may be used. In some embodiments, the anode layer may be an organic material such as polyaniline. The use of polyaniline is described in “Flexible light-emitting diodes made from soluble conducting polymer,” Nature, vol. 357, pp. 477-479 (11 Jun. 1992). Examples of suitable high work function metals and metal oxides include but are not limited to Au, Pt, or alloys thereof; ITO; IZO; and the like. In some embodiments, the anode layer can have a thickness in the range of about 1 nm to about 1000 nm.

A cathode may be a layer including a material having a lower work function than the anode layer. Examples of suitable materials for the cathode layer include those selected from alkali metals of Group 1, Group 2 metals, Group 12 metals including rare earth elements, lanthanides and actinides, materials such as aluminum, indium, calcium, barium, samarium and magnesium, and combinations thereof. Li-containing organometallic compounds, LiF, and Li₂O may also be deposited between the organic layer and the cathode layer to lower the operating voltage. Suitable low work function metals include but are not limited to Al, Ag, Mg, Ca, Cu, Mg/Ag, LiF/Al, CsF, CsF/Al or alloys thereof. In an embodiment, the cathode layer can have a thickness in the range of about 1 nm to about 1000 nm.

In some embodiments, the organic component may comprise at least one emissive layer comprising an emissive component, and optionally, a host, such as a compound described herein, a hole-transport material, an electron-transport material, or an ambipolar material. In some embodiments, the device is configured so that holes can be transferred from the anode to the emissive layer. In some embodiments, the device is configured so that electrons can be transferred from the cathode to the emissive layer. If present, the amount of the host in an emissive layer can vary. In one embodiment, the amount of a host in an emissive layer is in the range of from about 1% to about 99.9% by weight of the emissive layer. In another embodiment, the amount of a host in an emissive layer is in the range of from about 90% to about 99% by weight of the emissive layer. In another embodiment, the amount of a host in an emissive layer is about 97% by weight of the emissive layer. In some embodiments, the mass of the emissive component is about 0.1% to about 10%, about 1% to about 5%, or about 3% of the mass of the emissive layer. In some embodiments, the emissive layer may be a neat emissive layer, meaning that the emissive component is about 100% by weight of the emissive layer, or alternatively, the emissive layer consists essentially of emissive component.

The thickness of an emissive layer may vary. In one embodiment, an emissive layer has a thickness in the range of from about 1 nm to about 200 nm. In another embodiment, an emissive layer has a thickness in the range of about 1 nm to about 150 nm.

In another embodiment, an emissive layer, or a combination of emissive layers, may also be configured to emit white light.

In some embodiments, the organic component may further comprise a hole-transport layer disposed between the anode and the emissive layer. The hole-transport layer may comprise at least one hole-transport material. In some embodiments, the hole-transport material comprises at least one of an aromatic-substituted amine, a carbazole, a polyvinylcarbazole (PVK), e.g. poly(9-vinylcarbazole); polyfluorene; a polyfluorene copolymer; poly(9,9-di-n-octylfluorene-alt-benzothiadiazole); poly(paraphenylene); poly[2-(5-cyano-5-methylhexyloxy)-1,4-phenylene]; a benzidine; a phenylenediamine; a phthalocyanine metal complex; a polyacetylene; a polythiophene; a triphenylamine; an oxadiazole; copper phthalocyanine; 1,1-Bis(4-bis(4-methylphenyl)aminophenyl)cyclohexane; 2,9-Dimethyl-4,7-diphenyl-1,10-phenanthroline; 3,5-Bis(4-tert-butyl-phenyl)-4-phenyl [1,2,4]triazole; 3,4,5-Triphenyl-1,2,3-triazole; 4,4′,4′-tris(3-methylphenylphenylamino)triphenylamine (MTDATA); N,N′-bis(3-methylphenyl)N,N′-diphenyl-[1,1′-biphenyl]-4,4′-diamine (TPD); 4,4′-bis[N-(naphthyl)-N-phenyl-amino]biphenyl (α-NPD); 4,4′,4″-tris(carbazol-9-yl)-triphenylamine (TCTA); 4,4′-bis[N,N′-(3-tolyl)amino]-3,3′-dimethylbiphenyl (HMTPD); 4,4′-N,N′-dicarbazole-biphenyl (CBP); 1,3-N,N-dicarbazole-benzene (mCP); Bis[4-(p,p′-ditolyl-amino)phenyl]diphenylsilane (DTASi); 2,2′-bis(4-carb azolylphenyl)-1,1′-biphenyl (4CzPBP); N,N′N″-1,3,5-tricarbazoloylbenzene (tCP); N,N′-bis(4-butylphenyl)-N,N′-bis(phenyl)benzidine; or the like.

In some embodiments, the organic component may further comprise an electron-transport layer disposed between the cathode and the emissive layer. In some embodiments, the electron-transport layer may comprise a compound described herein. Other electron-transport materials may be included, such as 2-(4-biphenylyl)-5-(4-tert-butylphenyl)-1,3,4-oxadiazole (PBD); 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene; 3-phenyl-4-(1′-naphthyl)-5-phenyl-1,2,4-triazole (TAZ); 2,9-dimethyl-4,7-diphenyl-phenanthroline (bathocuproine or BCP); aluminum tris(8-hydroxyquinolate) (Alq3); and 1,3,5-tris(2-N-phenylbenzimidazolyl)benzene; 1,3-bis[2-(2,2′-bipyridine-6-yl)-1,3,4-oxadiazo-5-yl]benzene (BPY-OXD); 3-phenyl-4-(1′-naphthyl)-5-phenyl-1,2,4-triazole (TAZ), 2,9-dimethyl-4,7-diphenyl-phenanthroline (bathocuproine or BCP); and 1,3,5-tris[2-N-phenylbenzimidazol-z-yl]benzene (TPBI). In one embodiment, the electron transport layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl] benzene (TPBI), or a derivative or a combination thereof.

If desired, additional layers may be included in the light-emitting device. These additional layers may include an electron injection layer (EIL), a hole-blocking layer (HBL), an exciton-blocking layer (EBL), and/or a hole-injection layer (HIL). In addition to separate layers, some of these materials may be combined into a single layer.

In some embodiments, the light-emitting device can include an electron-injection layer between the cathode layer and the emissive layer. In some embodiments, the electron-injection layer may comprise a compound described herein. Other suitable electron injection materials may also be included, and are known to those skilled in the art. Examples of suitable material(s) that can be included in the electron injection layer include but are not limited to, an optionally substituted compound selected from the following: aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl] benzene (TPBI) a triazine, a metal chelate of 8-hydroxyquinoline such as tris(8-hydroxyquinoliate) aluminum, and a metal thioxinoid compound such as bis(8-quinolinethiolato) zinc. In one embodiment, the electron injection layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), or a derivative or a combination thereof.

In some embodiments, the device can include a hole-blocking layer, e.g., between the cathode and the emissive layer. Various suitable hole-blocking materials that can be included in the hole-blocking layer are known to those skilled in the art. Suitable hole-blocking material(s) include but are not limited to, an optionally substituted compound selected from the following: bathocuproine (BCP), 3,4,5-triphenyl-1,2,4-triazole, 3,5-bis(4-tert-butyl-phenyl)-4-phenyl-[1,2,4]triazole, 2,9-dimethyl-4,7-diphenyl-1,10-phenanthroline, and 1,1-bis(4-bis(4-methylphenyl)aminophenyl)-cyclohexane.

In some embodiments, the light-emitting device can include an exciton-blocking layer, e.g., between the emissive layer and the anode. In an embodiment, the band gap of the material(s) that comprise exciton-blocking layer is large enough to substantially prevent the diffusion of excitons. A number of suitable exciton-blocking materials that can be included in the exciton-blocking layer are known to those skilled in the art. Examples of material(s) that can compose an exciton-blocking layer include an optionally substituted compound selected from the following: aluminum quinolate (Alq₃), 4,4′-bis[N-(naphthyl)-N-phenyl-amino]biphenyl (α-NPD), 4,4′-N,N′-dicarbazole-biphenyl (CBP), and bathocuproine (BCP), and any other material(s) that have a large enough band gap to substantially prevent the diffusion of excitons.

In some embodiments, the light-emitting device can include a hole-injection layer, e.g., between the emissive layer and the anode. Various suitable hole-injection materials that can be included in the hole-injection layer are known to those skilled in the art. Exemplary hole-injection material(s) include an optionally substituted compound selected from the following: a polythiophene derivative such as poly(3,4-ethylenedioxythiophene (PEDOT)/polystyrene sulphonic acid (PSS), a benzidine derivative such as N,N,N′,N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), and a phthalocyanine metal complex derivative such as phthalocyanine copper. Hole-injection materials, while still being able to transport holes, may have a hole mobility substantially less than the hole mobility of conventional hole transport materials.

Light-emitting devices comprising the compounds described herein can be fabricated using techniques known in the art, as informed by the guidance provided herein. For example, a glass substrate can be coated with a high work functioning metal such as ITO which can act as an anode. After patterning the anode layer, a hole-injection and/or hole-transport layer may be deposited on the anode in that order. An emissive layer that includes an emissive component, can be deposited on the anode, the hole-transport layer, or the hole-injection layer. The emissive layer may contain a compound described herein, and/or a compound described herein may be part of an electron-transport layer and/or an electron-injecting layer, deposited in that order, or may be part of an electron-injecting and electron-transport layer. The cathode layer, comprising a low work functioning metal (e.g., Mg:Ag), can then be deposited, e.g., by vapor deposition or sputtering. The device may also contain an exciton-blocking layer, an electron blocking layer, a hole blocking layer, a second emissive layer, or other layers that can be added to the device using suitable techniques.

In some embodiments, the OLED is made by a wet process such as a process that comprises at least one of spraying, spin coating, drop casting, inkjet printing, screen printing, etc. Some embodiments provide a composition which is a liquid suitable for deposition onto a substrate. The liquid may be a single phase, or may comprise one or more additional solid or liquid phases dispersed in it. The liquid typically comprises a light-emitting compound, a host material described herein and a solvent.

### Synthetic Example

### Synthesis procedure for ET-1, ET-2, ET-3, ET-4

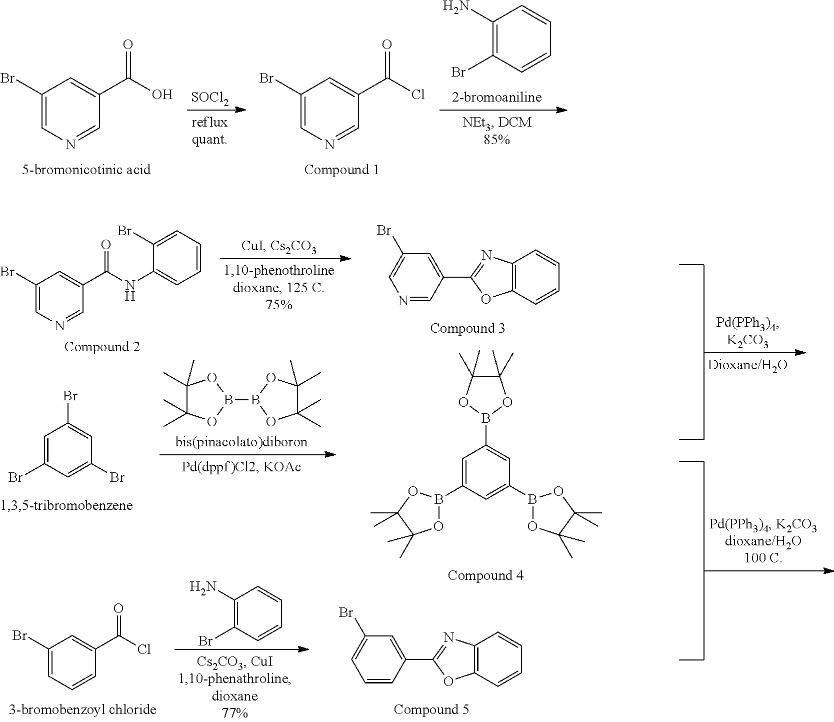

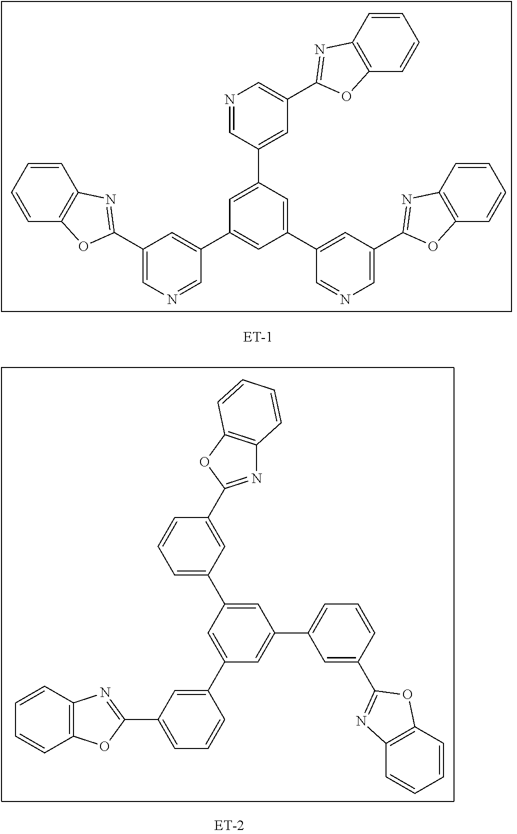

5-Bromonicotinoyl chloride (1): To a mixture of 5-bromonicotinic acid (10 g) in thionyl chloride (25 mL) was added anhydrous DMF (0.5 mL). The mixture was refluxed overnight, cooled to room temperature, and excess thionyl chloride was removed under reduced pressure. A white solid 1 (11 g) was obtained, which was used for the next step without further purification.

5-bromo-N-(2-bromophenyl)nicotinamide (2): A mixture of 5-bromonicotinoyl chloride (1) (7.5 g, 33 mmol), 2-bromoaniline (5.86 g, 33 mmol) and triethylamine (NEt₃) (14 mL, 100 mmol) in anhydrous dichlormethane (DCM) (100 mL) was stirred under argon overnight. The resulting mixture was worked up with water and extracted with dichloromethane (200 mL×2). The organic phase was collected and dried over Na₂SO₄. After the organic phase was concentrated to 150 mL, a white crystalline solid 2 crashed out. Filtration and washing with hexanes gave a white solid (10.0 g, 85% yield).

2-(5-bromopyridin-3-yl)benzo[d]oxazole (3): A mixture of 5-bromo-N-(2-bromophenyl)nicotinamide (2) (3.44 g, 9.7 mmol), CuI (0.106 g, 0.56 mmol), Cs₂CO₃ (3.91 g, 12 mmol) and 1,10-phenathroline (0.20 g, 1.12 mmol) in anhydrous 1,4-dioxane (50 mL) was heated at 100° C. overnight. After being cooled to room temperature, the mixture was poured into ethyl acetate (200 mL) and washed with water. The aqueous phase was extracted with ethyl acetate (200 mL×2), and the organic phase was collected and dried over Na₂SO₄, purified by flash chromatography (silica gel, hexanes/ethyl acetate 3:1) to give a light yellow solid 3 (2.0 g, 75% yield).

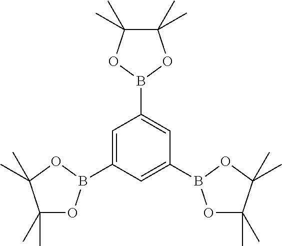

1,3,5-tris(4,4,5,5-tetramethyl-1,3,2-dioxaborolan-2-yl)benzene (4): A mixture of 1,3,5-tribromobenzene (7.96 g, 25.3 mmol), bis(pinacolato)diboron (21.2 g, 83.5 mmol), [1,1′-Bis(diphenylphosphino)ferrocene]dichloropalladium (2.78 g, 3.79 mmol), potassium acetate (22.3 g, 228 mmol) in anhydrous 1,4-dioxane (400 mL) was degassed for 80 minutes. Mixture was then heated to 80° C. overnight under argon. After cooling to room temperature, the remaining solids were filtered off. The filtrate was dried under vacuum, redissolved in methylene chloride (400 mL) then washed with water (2×300 mL) and brine (300 mL). Organic layer was dried over sodium sulfate and loaded onto silica gel. A silica plug (11% ethyl acetate in hexanes) and precipitation from methylene chloride/methanol gave 4 (9.76 g, 88% yield).

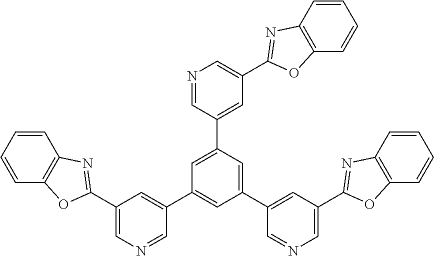

1,3,5-tris(5-(benzo[d]oxazol-2-yl)pyridin-3-yl)benzene (ET-1): A mixture of 1,3,5-tris(4,4,5,5-tetramethyl-1,3,2-dioxaborolan-2-yl)benzene (4) (456 mg, 1.0 mmol), 2-(5-bromopyridin-3-yl)benzo[d]oxazole (822 mg, 3.0 mmol), Pd(PPh₃)₄ (180 mg, 0.16 mmol) and K₂CO₃ (828 mg, 6.0 mmol) in 1,4-dioxane (30 mL) and water (6 mL) was degassed, then heated at 90° C. overnight. A light yellow precipitate was filtered and washed with methanol to give an off white solid (ET-1) (0.62 g, 95% yield).

2-(3-bromophenyl)benzo[d]oxazole (5): A mixture of 3-bromobenzoyl chloride (10.0 g, 45.6 mmol), 2-bromoaniline (7.91 g, 46 mmol), Cs₂CO₃ (30 g, 92 mmol), CuI (0.437 g, 2.3 mmol) and 1,10-phenathroline (0.829 g, 4.6 mmol) in anhydrous 1,4-dioxane (110 mL) was heated at 120° C. for 8 h. After the mixture was cooled to room temperature, it was poured into ethyl acetate (300 mL) and worked up with water (250 mL). The aqueous solution was extracted with dichloromethane (300 mL). The organic phase was collected and combined, dried over Na₂SO₄. Purification by a short silica gel column (hexanes/ethyl acetate 3:1) gave a solid which was washed with hexanes to give a light yellow solid (5) (9.54 g, 76% yield).

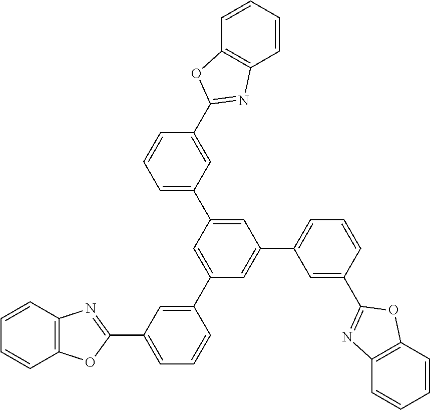

2,2′-(5′-(3-(benzo[d]oxazol-2-yl)phenyl)-[1,1′:3′,1″-terphenyl]-3,3″-diyl)bis(benzo[d]oxazole) (ET-2): A mixture of 1,3,5-tris(4,4,5,5-tetramethyl-1,3,2-dioxaborolan-2-yl)benzene (4) (1.824 g, 4.0 mmol), 2-(3-bromophenyl)benzo[d]oxazole (5) (3.28 g, 12 mmol), Pd(PPh₃)₄ (0.74 g, 0.64 mmol) and K₂CO₃ (3.31 g, 24 mmol) in 1,4-dioxane (100 mL) and water (20 mL) was degassed then heated at 95° C. under argon overnight. After cooled to room temperature, filtration and washing with methanol gave a white solid (ET-2) (2.62 g, quantitative yield).

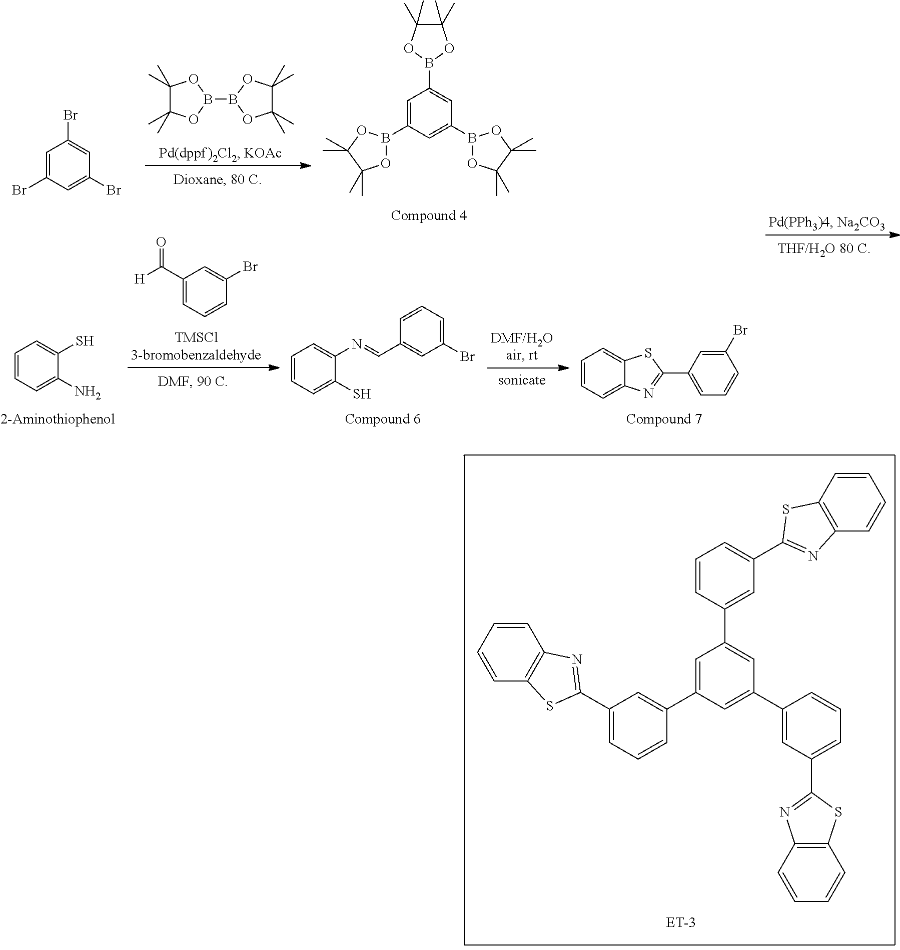

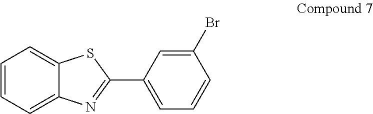

2-(3-bromophenyl)benzo[d]thiazole (7): 2-Aminothiophenol (5.0 g, 39.9 mmol) and 3-bromobenzaldehyde (7.39 g, 39.9 mmol) was dissolved in anhydrous DMF. Trimethylsilylchloride (10.84 g, 99.8 mmol) was added dropwise. The resulting solution was heated at 90° C. overnight under argon. After cooling to room temperature, the mixture was poured into water (100 mL). Mixture was then sonicated in open air for 3 hours, poured into water (200 mL), and extracted with methylene chloride (2×200 mL). Organic washes were combined, washed with brine (200 mL), and dried over sodium sulfate. Flash column (gradient of 3 to 9% ethyl acetate in hexanes) and additional silica plug (5% ethyl acetate in hexanes) gave 4.09 g of material 7 in 35% yield.

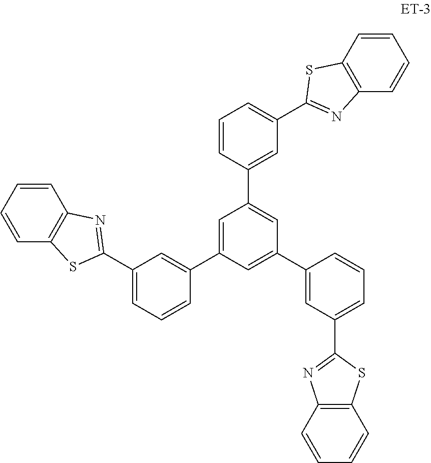

1,3,5-tris(1′-benzo[d]thiazol-2″-yl-phenyl-3′-yl)-benzene (ET-3): A mixture of 1,3,5-tris(4,4,5,5-tetramethyl-1,3,2-dioxaborolan-2-yl)benzene (4) (500 mg, 1.14 mmol), 2-(3-bromophenyl)benzo[d]thiazole (7) (1.15 g, 3.98 mmol), tetrakis(triphenylphosphine)palladium (140 mg, 0.12 mmol), sodium carbonate (1.51 g, 14.2 mmol), THF (25 mL), and water (14 mL) was degassed for 25 minutes. The mixture was heated at reflux (80° C.) overnight under argon. After cooling, the mixture was poured into ethyl acetate (125 mL) then washed with saturated sodium bicarbonate solution (100 mL), water (100 mL), and brine (100 mL). A flash column (gradient of 5 to 10% acetone in hexanes) and precipitation from methylene chloride/methanol gave 611 mg of material (ET-3) in 76% yield.

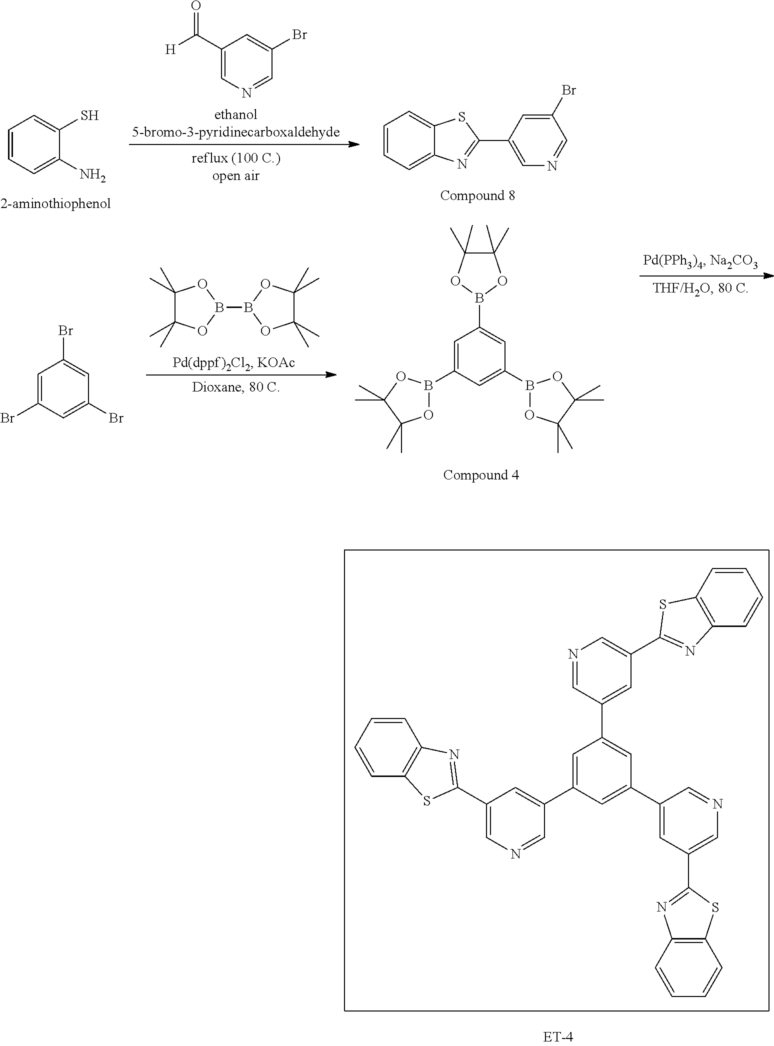

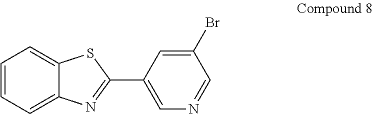

2-(5-bromopyridin-3-yl)benzo[d]thiazole (8): To a mixture of 2-aminothiophenol (500 mg, 3.99 mmol) and 5-bromo-3-pyridinecarboxaldehyde (743 mg, 3.99 mmol) was added ethanol (10 mL). The mixture was then heated at reflux (100° C.) overnight under ambient air. After cooling, mixture was dried under vacuum then redissolved in methylene chloride (100 mL). The methylene chloride solution was washed with water (100 mL) and brine (50 mL), and dried over sodium sulfate. The crude material was run through a plug of silica (16% ethyl acetate in hexanes), and precipitated from methanol to give 564 mg of material (8) in 49% yield.

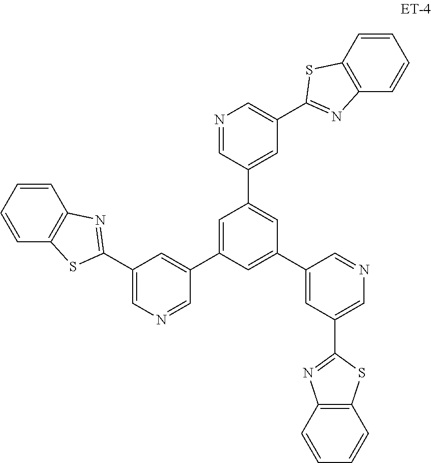

1,3,5-tris(5′-(benzo[d]thiazol-2″-yl)pyridin-3′-yl)benzene (ET-4): A mixture of 2-(5-bromopyridin-3-yl)benzo[d]thiazole (8) (4.0 g, 13.7 mmol), 1,3,5-tris(4,4,5,5-tetramethyl-1,3,2-dioxaborolan-2-yl)benzene (4) (1.83 g, 4.16 mmol), tetrakis(triphenylphosphine)palladium (474 mg, 0.411 mmol), sodium carbonate (5.18 g, 48.9 mmol), THF (100 mL), and water (60 mL) was degassed for 20 minutes. The mixture was heated at reflux (85° C.) overnight under argon. After cooling to room temperature, the reaction mixture was placed into a separatory funnel and the aqueous layer was removed. The organic phase (containing solids) was then filtered and washed with water, methanol, acetone and THF. Remaining solids were dried to give 2.70 g of product (ET-4) in 92% yield.

### Example of OLED Device Configuration and Performance

Fabrication of white light-emitting device: the ITO coated glass substrates were cleaned by ultrasound in deionized (DI)-water, acetone, and consecutively in 2-propanol, then baked at 110° C. for about 3 hours, followed by treatment with oxygen plasma for about 30 min. A layer of PEDOT: PSS (Baytron P purchased from H.C. Starck) was spin-coated at about 6000 rpm onto the pre-cleaned and O₂-plasma treated (ITO)-substrate and annealed at about 200° C. for about 30 min, yielding a thickness of around 20 nm. In a glove-box hosted vacuum deposition system at a pressure of about 10⁻⁷ torr (1 torr=133.322 Pa), DTASi was first deposited on top of PEDOT/PSS layer at deposition rate of about 1 Å/s, yielding a 40 nm thick film. Then, for the first emissive layer (EM-1), the Host-1 and Blue emitter (FirPic, 12 wt %) were co-deposited to a 5 nm thickness. Then, for the second emissive layer (EM-2), the Host-2 with Yellow (YE-1, 4 wt %) and Red (Ir(piq)₂acac, 0.5 wt %) emitter were co-deposited to a 8 nm thickness.

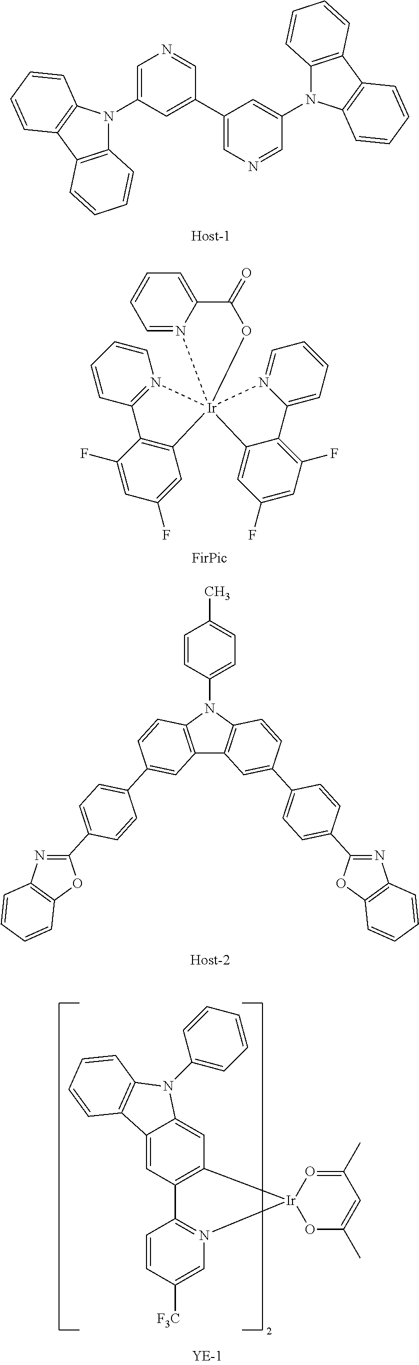

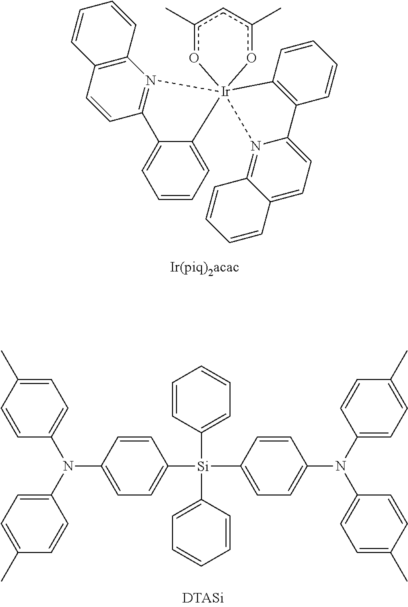

Next, the electron transport layer (ETL) was deposited, either as ET-2 or as 1,3,5-tris(N-phenylbenzimidizol-2-yl)benzene (TPBI [Comparative Example]), at a deposition rate around 1 Å/s to form a 40 nm thick film. LiF (0.5 nm) and Al (100 nm) were then deposited successively at deposition rates of about 0.05 and about 2 Å/s, respectively. Each individual device had a surface area of about 0.08 cm². All Electron luminescence spectra were measured MCPD spectrometer and I-V-L characteristics were taken with a Keithley 2400 and 2000 Meter and Si-photo diode. All device operation was performed in air after encapsulation in glove box.

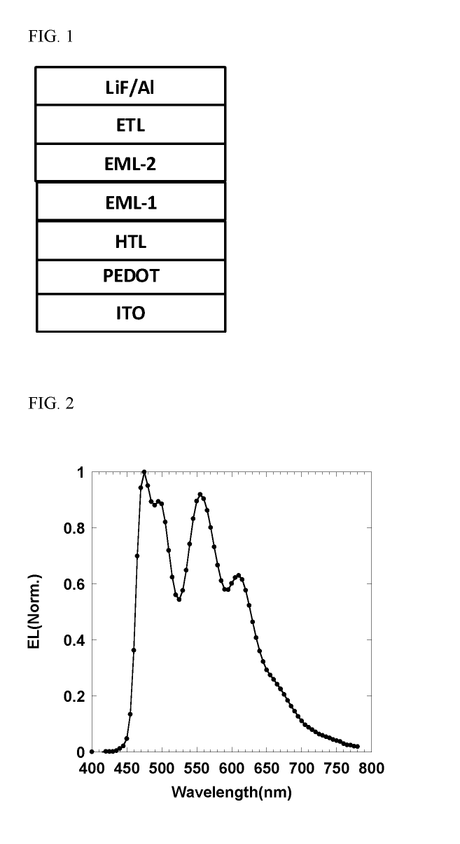

An exemplary configuration of the device comprising ET-2 is shown in FIG. 1. The device comprises following layers in the order given: an ITO anode, a PEDOT hole-injection layer, a hole-transport layer (HTL), a first emissive layer (EM-1), a second emissive layer (EM-2), an electron-transport layer (ETL), and a LiF/Al cathode.

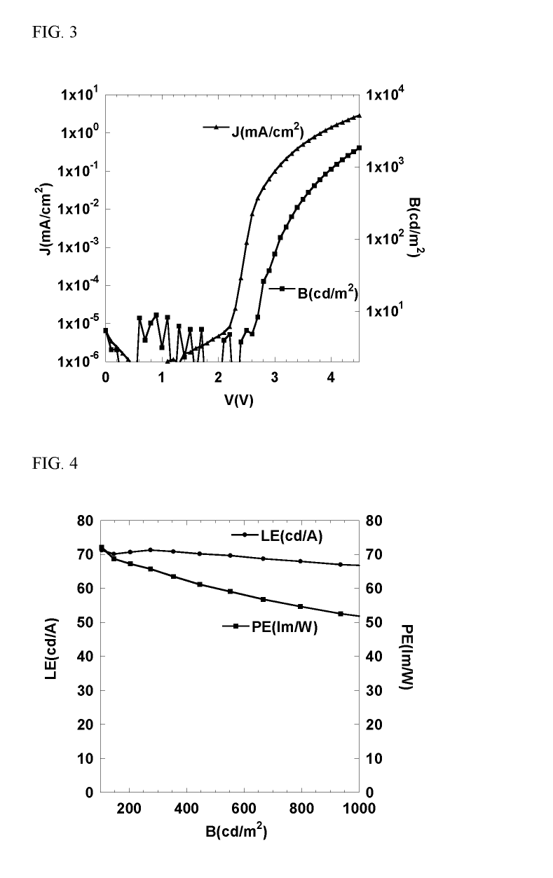

FIG. 2 shows the electroluminescence spectrum of the device comprising ET-2, which shows strong emission throughout a broad portion the visible region, with the CIE coordinate of (0.35, 0.43) and CRI of 68. FIG. 3 shows current density and luminance as a function of the driving voltage of the device comprising ET-2. FIG. 4 shows the device current and power efficiency as a function of device brightness. Table-1 shows device performance of using ET-2 as ETM, in comparison with using TPBI (a common electron-transport material) as ETM in a same device configuration. These data show that ET-2, one embodiment of the compounds described herein, provides a device with similar or better performance, in terms of efficiency and color, than a common electron-transport material.

TABLE 1

| ETM | V @1000 nit | CIE (x, y) | CRI | PE (lm/W) | LE (cd/A) | EQE) |
|---|---|---|---|---|---|---|
| TPBI | 3.9 | 0.38, 0.44 | 70 | 50 | 62 | 25.6% |
| ET-2 | 4.1 | 0.35, 0.43 | 68 | 51 | 66 | 28% |

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. A compound represented by a formula: 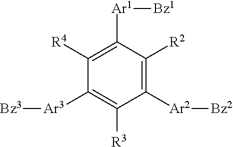
wherein Ar¹, Ar², and Ar³ are independently m-phenylene or m-pyridinylene, optionally substituted with 1 or 2 substituents selected from the group consisting of: F, Cl, CF₃, C₁₋₆ alkyl, and C₁₋₆ alkoxy;
Bz¹, Bz², and Bz³ are independently optionally substituted benzooxazol-2-yl or benzothiazol-2-yl, optionally substituted with 1 or 2 substituents selected from the group consisting of: F, Cl, CF₃, C₁₋₆ alkyl, and C₁₋₆ alkoxy; and
R², R³, and R⁴ are independently selected from the group consisting of: H, F, Cl, CF₃, C₁₋₆ alkyl, and C₁₋₆ alkoxy.

2. The compound of claim 1, wherein R², R³, and R⁴ are H.

3. The compound of claim 2, wherein Ar¹, Ar², and Ar³ are unsubstituted.

4. The compound of claim 1, wherein Ar¹, Ar², and Ar³ are unsubstituted.

5. The compound of claim 1, wherein Bz¹, Bz², and Bz³ are unsubstituted.

6. The compound of claim 1, wherein Ar¹, Ar², and Ar³ are unsubstituted.

7. The compound of claim 1, selected from the group consisting of:

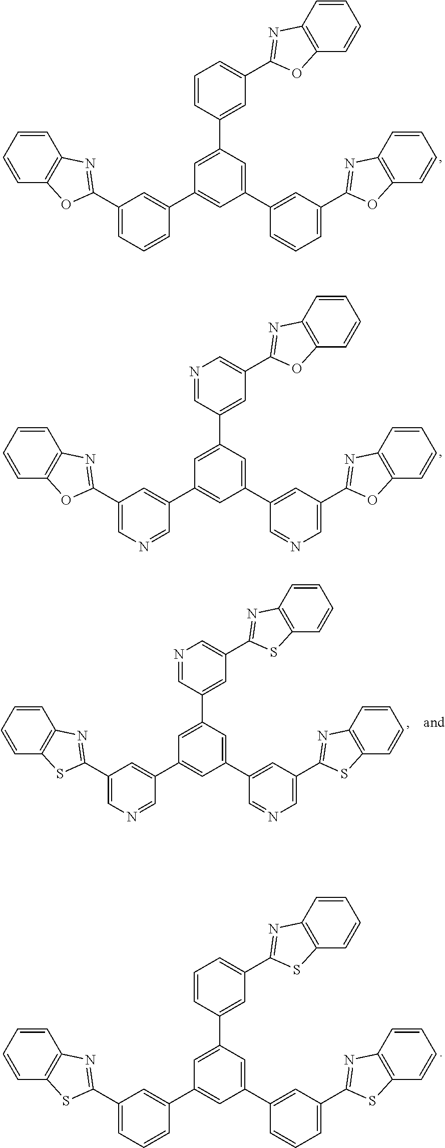

8. A compound represented by a formula: 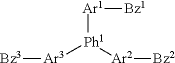
wherein Ph¹ is optionally substituted phenyl;
Ar¹, Ar², and Ar³ are independently optionally substituted m-phenylene or m-pyridinylene; and
Bz¹, Bz², and Bz³ are independently optionally substituted benzooxazol-2-yl or benzothiazol-2-yl.

9. The compound of claim 8, wherein Ph¹ has a 1, 3, 5-substitution pattern.

10. The compound of claim 9, wherein Ph¹ is unsubstituted.

11. The compound of claim 9, wherein Ar¹ is unsubstituted.

12. The compound of claim 9, wherein Ar² is unsubstituted.

13. The compound of claim 9, wherein Ar³ is unsubstituted.

14. The compound of claim 9, wherein Bz¹ is unsubstituted.

15. The compound of claim 9, wherein Bz² is unsubstituted.

16. The compound of claim 9, wherein Bz³ is unsubstituted.

17. An organic light-emitting device comprising a compound of claim 8.

18. The device of claim 17, wherein Ar¹ is unsubstituted.

19. The device of claim 17, wherein Ar² is unsubstituted.

20. The device of claim 17, wherein Ar³ is unsubstituted.

21. The device of claim 17, wherein the compound is selected from the group consisting of: [chemical structure]
